SEQUENCE LISTING (25 sequences)

SEQ ID NO: 1 - protein, 183 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 2 - DNA, 551 bp, Homo sapiens
acagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtag

SEQ ID NO: 3 - protein, 207 aa, Mus musculus
METDTLLLWVLLLWVPGSTGDAAHTHQDFQPVLHLVALNTPLSGGMRGIRGADFQCFQQARAVGLSGTFRAFLSSRLQDLYSIVRRADRGSVPIVNLKDEVLSPSWDSLFSGSQGQLQPGARIFSFDGRDVLRHPAWPQKSVWHGSDPSGRRLMESYCETWRTETTGATGQASSLLSGRLLEQKAASCHNSYIVLCIENSFMTSFSK

SEQ ID NO: 4 - DNA, 624 bp, Mus musculus
atggagacagacacactcctgctatgggtactgctgctctgggttccaggttccactggtgacgcggcccatactcatcaggactttcagccagtgctccacctggtggcactgaacacccccctgtctggaggcatgcgtggtatccgtggagcagatttccagtgcttccagcaagcccgagccgtggggctgtcgggcaccttccgggctttcctgtcctctaggctgcaggatctctatagcatcgtgcgccgtgctgaccgggggtctgtgcccatcgtcaacctgaaggacgaggtgctatctcccagctgggactccctgttttctggctcccagggtcaagtgcaacccggggcccgcatcttttcttttgacggcagagatgtcctgagacacccagcctggccgcagaagagcgtatggcacggctcggaccccagtgggcggaggctgatggagagttactgtgagacatggcgaactgaaactactggggctacaggtcaggcctcctccctgctgtcaggcaggctcctggaacagaaagctgcgagctgccacaacagctacatcgtcctgtgcattgagaatagcttcatgacctctttctccaaatag

SEQ ID NO: 5 - protein, 8 aa, Homo sapiens
APQQEALA

SEQ ID NO: 6 - DNA, 38 bp, Artificial Sequence
  Description of Artificial Sequence Synthetic primer
actggtgacgcggcccatactcatcaggactttcagcc

SEQ ID NO: 7 - DNA, 32 bp, Artificial Sequence
  Description of Artificial Sequence Synthetic primer
aagggctatcgatctagctggcagaggcctat

SEQ ID NO: 8 - DNA, 20 bp, Artificial Sequence
  Description of Artificial Sequence Synthetic primer
cactgcttactggcttatcg

SEQ ID NO: 9 - DNA, 29 bp, Artificial Sequence
  Description of Artificial Sequence Synthetic primer
ctgatgagtatgggccgcgtcaccagtgg

SEQ ID NO: 10 - DNA, 32 bp, Artificial Sequence; identical to SEQ ID NO: 7
  Description of Artificial Sequence Synthetic primer

SEQ ID NO: 11 - DNA, 35 bp, Artificial Sequence
  Description of Artificial Sequence Synthetic primer
gatctctagaccaccatgcatactcatcaggactt

SEQ ID NO: 12 - DNA, 30 bp, Artificial Sequence
  Description of Artificial Sequence Synthetic primer
actggagaaagaggtttatctagctactag

SEQ ID NO: 13 - protein, 18 aa, Adenovirus
MRYMILGLLALAAVCSAA

SEQ ID NO: 14 - DNA, 96 bp, Artificial Sequence
  Description of Artificial Sequence Synthetic primer
gatctctagaccaccatgaggtacatgattttaggcttgctcgcccttgcggcagtctgcagcgcggcccatactcatactcatcaggactttcag

SEQ ID NO: 15 - DNA, 29 bp, Artificial Sequence
  Description of Artificial Sequence Synthetic primer
atcgatcatactcatcaggactttcagcc

SEQ ID NO: 16 - DNA, 29 bp, Artificial Sequence
  Description of Artificial Sequence Synthetic primer
gcggccgcctatttggagaaagaggtcat

SEQ ID NO: 17 - DNA, 23 bp, Artificial Sequence
  Description of Artificial Sequence Synthetic primer
tttttttttcagtgtaaaaggtc

SEQ ID NO: 18 - DNA, 19 bp, Artificial Sequence
  Description of Artificial Sequence Synthetic primer
cagatgacatcctggccag

SEQ ID NO: 19 - DNA, 22 bp, Artificial Sequence
  Description of Artificial Sequence Synthetic primer
ctatacaggaaagtatggcagc

SEQ ID NO: 20 - DNA, 118 bp, Artificial Sequence
  Description of Artificial Sequence Synthetic oligonucleotide
gccaagcttccatgagggcctggatcttctttctcctttgcctggccgggagggctctggcagcccctcagcaagaagcgctcgctcacagccaccgcgacttccagccggtgctcca

SEQ ID NO: 21 - DNA, 123 bp, Artificial Sequence
  Description of Artificial Sequence Synthetic oligonucleotide
ccaggtggagcaccggctggaagtcgcggtggctgtgagcgagcgcttcttgctgaggggctgccagagccctcccggccaggcaaaggagaaagaagatccaggccctcatggaagcttggc

SEQ ID NO: 22 - DNA, 28 bp, Artificial Sequence
  Description of Artificial Sequence Synthetic primer
gcgcatgtcgacagaatatgggccaaac

SEQ ID NO: 23 - DNA, 28 bp, Artificial Sequence
  Description of Artificial Sequence Synthetic primer
gcgctactgcagagctaatgagctacac

SEQ ID NO: 24 - DNA, 27 bp, Artificial Sequence
  Description of Artificial Sequence Synthetic primer
ccggctagcttaagggtggcgaccggt

SEQ ID NO: 25 - DNA, 27 bp, Artificial Sequence
  Description of Artificial Sequence Synthetic primer
gcttcgaacgcgtagcggccaaccctc

(19) United States
(12) Patent Application Publication
(10) Pub. No.: US 2006/0251621 A1
(43) Pub. Date: Nov. 9, 2006

(54) Ocular gene therapy

(76) Inventors: Peter Anthony Campochiaro, Baltimore, MD (US); Michael Kaleko, Rockville, MD (US)

Correspondence Address: BELL, BOYD, & LLOYD LLC, P. O. BOX 1135, CHICAGO, IL, 60690-1135, US

(21) Appl. No.: 10/529,428

(22) PCT Filed: Sep. 26, 2003
(86) PCT No.: PCT/EP03/10725

§ 371 Date: Mar. 24, 2006

Related U.S. Application Data
(60) Provisional application No. 60/414,048, filed on Sep. 27, 2002.

(51) Int. Cl. A61K 48/00; C12N 15/861

(52) U.S. Cl. 424/93.2; 435/456

(57) ABSTRACT

Methods are provided for the delivery of a protein to the retina of a subject in need of such delivery, comprising periocularly injecting the individual with an effective amount of a viral vector comprising a protein-encoding nucleic acid.

### FIELD OF THE INVENTION

The present invention relates to methods for treating disorders of the retina using gene therapy.

### SUMMARY OF THE INVENTION

The present invention provides methods for the delivery of a protein to the retina of a subject in need of such delivery, comprising periocularly injecting the subject with a composition comprising an effective amount of a vector, such as a viral vector, comprising a protein-encoding nucleic acid. The vector, once injected, is capable of effecting the expression of the protein in the retina of the subject. The protein can be, e.g., an endostatin, e.g., with the amino acid sequence set forth in SEQ ID NO:1, a derivative of the polypeptide with the amino acid sequence set forth in SEQ ID NO:1, or a variant of the polypeptide with the amino acid sequence set forth in SEQ ID NO:1. The viral vector can be, e.g., selected from an adenovirus, an adeno-associated virus, a retrovirus, and a lentivirus.

In a preferred aspect, endostatin is endostatin or an active fragment of endostatin.

Examples of such active fragments and variants are set forth, e.g., in U.S. Pat. No. 6,174,861, the disclosure of which is incorporated herein in its entirety.

The methods of the present invention are useful for the treatment of retinal disorders including, e.g., retinal detachment, diabetic retinopathy, retinal neovascularization, choroidal neovascularization, and retinal edema, including macular edema.

### DETAILED DESCRIPTION OF THE INVENTION

The present invention provides a method for the prophylactic and treatment of retinal disorders in a subject, e.g., retinal detachment, diabetic retinopathy, retinal neovascularization, choroidal neovascularization, and retinal edema, including macular edema, by periocular injection of a composition comprising a vector comprising a nucleic acid encoding a protein, preferably a protein with antiangiogenic activity or which prevents breakdown of the blood-retinal barrier.

In one embodiment, the vector is a viral vector. Viral vectors which may be employed include RNA virus vectors (such as retroviral vectors, such as MOMLV-based vectors, or lentiviral vectors, including, but not limited to, HIV and BIV) and DNA virus vectors (such as adenoviral vectors, adeno associated virus vectors, Herpes Virus vectors, and vaccinia virus vectors).

“Treatment” encompasses both prophylactic and treatment. By “prophylactic” is meant the protection, in whole or in part, against retinal disorders. By “therapeutic” is meant the amelioration of a retinal disorder itself, and the protection, in whole or in part, against further retinal disorders or exacerbation of an existing disorder. The present invention is particularly useful in the treatment of retinal detachment, diabetic retinopathy, retinal neovascularization, choroidal neovascularization, and retinal edema, including macular edema.

As used herein, “a retinal disorder-inhibiting effective amount” of a protein is that amount of a protein that will cause any or all of: 1) a decrease in retinal vascular permeability; 2) a decrease in retinal thickness; 3) an absolute inhibition of or a decrease in the degree of retinal detachment; 4) a decrease in the absolute amount of retinal neovascularization; 5) a decrease in the rate of retinal neovascularization; 6) a cessation of progression of retinal neovascularization; 7) a decrease in the absolute amount of choroidal neovascularization; 8) a decrease in the rate of choroidal neovascularization; or 9) a cessation of progression of choroidal neovascularization.

As used herein, the term “viral vector” is used according to its art-recognized meaning. It refers to a nucleic acid vector construct, which includes at least one element of viral origin and may be packaged into a viral vector particle. The viral vector particles may be utilized for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, adenoviral vectors, and adeno-associated viral (AAV) vectors.

The term “adenoviral particle” is to be understood broadly as meaning infectious viral particles that are formed when an adenoviral vector of the invention is transduced into an appropriate cell or cell line for the generation of infectious particles. As used herein, the term “adenovirus” or “adenoviral particle” is used to include any and all viruses that may be categorized as an adenovirus, including any adenovirus that infects a human or an animal, including all groups, subgroups, and serotypes. Preferably, such adenoviruses are ones that infect human cells. Such adenoviruses may be wild-type or may be modified in various ways known in the art or as disclosed herein. Such modifications include modifications to the adenovirus genome that is packaged in the particle in order to make an infectious virus. Such modifications include deletions known in the art, such as deletions in one or more of the E1, E2a, E2b, E3, or E4 coding regions. Such modifications also include deletions of all of the coding regions of the adenoviral genome. Such adenoviruses are known as “gutless” adenoviruses. This “gutless” adenoviral vector includes an adenoviral 5′ ITR, an adenoviral packaging signal and an adenoviral 3′ ITR (Sandig, et al., PNAS, 97(3):1002-1007 (2000); Reddy, et al., Mol. Ther., 5(1):63-73 (2002)). The vector contains from about 26 kb to about 38 kb, preferably 28 kb to 32 kb, and may include one or more genomic elements. The terms also include replication-conditional adenoviruses; that is, viruses that replicate in certain types of cells or tissues but not in other types. These include the viruses disclosed in U.S. Pat. No. 5,998,205, issued Dec. 7, 1999 to Hallenbeck et al. and U.S. Pat. No. 5,801,029, issued Sep. 1, 1998 to McCormick, the disclosures of both of which are incorporated herein by reference in their entirety. Such viruses are sometimes referred to as cytolytic or cytopathic viruses (or vectors), and, if they have such an effect on neoplastic cells, are referred to as oncolytic viruses (or vectors). These adenoviral vectors may be produced in adenoviral packaging cells as disclosed above. The preferred packaging cells are those that have been designed to limit homologous recombination that could lead to wild-type adenoviral particles. Such cells are disclosed in U.S. Pat. No. 5,994,128, issued Nov. 30, 1999 to Fallaux, et al., and U.S. Pat. No. 6,033,908, issued Mar. 7, 2000 to Bout, et al. The packaging cell known as PER.C6, which is disclosed in these patents, is particularly preferred.

A preferred protein is an endostatin. Other proteins that can be employed in the methods of the invention include, but are not limited to, soluble vascular endothelial growth factor receptor, pigment epithelium-derived factor, angiostatin (plasminogen fragment), rod-derived cone viability factor, antiangiogenic antithrombin III, cartilage-derived inhibitor (CDI), CD59 complement fragment, fibronectin fragment, Gro-beta, a heparinase, human chorionic gonadotropin (hCG), an interferon, interferon inducible protein (IP-10), interleukin-12, kringle 5 (plasminogen fragment), metalloproteinase inhibitors (TIMPs), placental ribonuclease inhibitor, plasminogen activator inhibitor, platelet factor-4 (PF4), prolactin 16 kD fragment, proliferin-related protein (PRP), thrombospondin-1 (TSP-1), transforming growth factor-beta (TGF-b), vasculostatin, and vasostatin (calreticulin fragment).

The term “DNA sequence encoding endostatin” as used herein means DNA which encodes a full-length endostatin or an active fragment, derivative, or analog of endostatin, e.g., such DNA may be a full-length gene encoding a full-length endostatin, or a truncated gene, or a mutated gene encoding a fragment or derivative or analog of such endostatin which has endostatin activity. The term “DNA sequence” refers generally to a polydeoxyribonucleotide molecule and more specifically to a linear series of deoxyribonucleotides connected one to the other by phosphodiester bonds between the 3′ and 5′ carbons of the adjacent pentoses.

DNA sequences encoding endostatin and fragments or derivatives thereof are shown and described in U.S. Pat. No. 5,854,205, which is incorporated by reference herein in its entirety. DNA sequences encoding other antiangiogenic proteins are known to those of skill in the art and are generally available in public sequence databases.

The term “endostatin” refers to a protein that is preferably 18 kDa to 20 kDa in size as determined by non-reduced and reduced gel electrophoresis, respectively. The term endostatin also includes active precursor forms of the 18 kDa to 20 kDa protein. The amino acid sequence of full-length human endostatin is set forth in SEQ ID NO:1. The nucleic acid sequence encoding human endostatin is set forth in SEQ ID NO:2. The amino acid sequence of mouse endostatin, plus the mouse Ig kappa leader sequence, is set forth in SEQ ID NO:3. The nucleic acid sequence encoding mouse endostatin with the mouse Ig kappa leader sequence is set forth in SEQ ID NO:4.

The term endostatin also includes fragments of the 18 kDa to 20 kDa protein and modified proteins and peptides that have a substantially similar amino acid sequence, and which are capable inhibiting proliferation of endothelial cells. For example, silent substitutions of amino acids, where the replacement of an amino acid with a structurally or chemically similar amino acid does not significantly alter the structure, conformation or activity of the protein, is well known in the art. Such silent substitutions are intended to fall within the scope of the appended claims.

It will be appreciated that the term “endostatin” includes shortened polypeptides where one or more amino acid is removed from either or both ends of full-length endostatin (i.e., the polypeptide with SEQ ID NO: 1), or from an internal region of the protein, yet the resulting molecule remains effective to inhibit endothelial cell proliferation and /or to treat retinal detachment, retinal edema, and/or ocular neovascularization. Such shortened polypeptides are referred to herein as “fragments.” The term “endostatin” also includes lengthened proteins or peptides where one or more amino acid is added to either or both ends of endostatin, or to an internal location in the protein, yet the resulting molecule retains endothelial proliferation inhibiting activity. Such molecules, for example with tyrosine added in the first position, are useful for labeling, using, e.g., 1251. Labeling with other radioisotopes may be useful in providing a molecular tool for destroying the target cell containing endostatin receptors. Labeling with “targeting” molecules such as ricin may provide a mechanism for destroying cells with endostatin receptors. Lengthened endostatin polypeptides, or endostatin polypeptides that have been covalently modified, are collectively referred to herein as “derivatives” of endostatin.

“Substantial sequence homology”, as used herein, means at least approximately 70% homology between amino acid residue sequence in the endostatin analog sequence and that of endostatin, preferably at least approximately 80% homology, more preferably at least approximately 90% homology.

Also included in the definition of the term “endostatin” are modifications of the endostatin protein and its peptide fragments. Such modifications include substitutions of naturally occurring amino acids at specific sites with other molecules, including but not limited to naturally and non-naturally occurring amino acids. Such substitutions may modify the bioactivity of endostatin and produce biological or pharmacological agonists or antagonists. Such modified polypeptides are referred to herein as “variants.” Variants, derivatives, and fragments of endostatin that have been shown to have antitumor effects and/or antiangiogenic effects are known and have been reported, e.g., in published international patent application numbers WO0067771, WO0063249, WO9931616, WO9929855, and WO9948924, the disclosures of which are incorporated by reference herein in their entirety. Such variants, derivatives, and fragments of endostatin are also useful in the methods of the present invention.

In one embodiment, viral vectors of the present invention are delivered periocularly to the eye of an animal in vivo, e.g., by injection into the conjunctiva, muscle, sclera, fascia, adipocytes, or the optic nerve.

Preferred vectors include retroviral vectors and lentiviral vectors (See, Coffin, et al., “Retroviruses”, (1997) Chapter 9 pp; 437473 Cold Spring Harbor Laboratory Press.). Vectors useful in the invention are produced recombinantly by procedures already taught in the art. WO94/29438, WO97/21824, WO97/21825, WO01/44458 and U.S. Pat. No. 5,672,510 describe the construction of retroviral and lentiviral packaging plasmids and packaging cell lines. Exemplary vectors include pCMV mammalian expression vectors, such as pCMV6b and pCMV6c (Chiron Corp.), pSFFV-Neo, and pBluescript-Sk+. Non-limiting examples of useful retroviral vectors are those derived from murine, avian, or primate retroviruses. Common retroviruses are those based on the Moloney murine leukemia virus (MoMLV-vector). Other MoMLV derived vectors include, Lmily, LINGFER, MINGFR and MINT (Chang et al., Blood 92:1-11 (1998)). Further vectors include those based on Gibbon ape leukemia virus (Galv) and Moloney murine sarcoma virus (MoMSV) and spleen focus forming virus (SFFV). Vectors derived from the murine stem cell virus (MESV) include MESV-MiLy, (Agarwal et al., J. of Virology, 72:3720-3728, (1998)). Non-limiting examples of lentiviral vectors include those derived from Equine Infectious Anemia Virus (EIAV), Simian Immunodeficiency Virus (SIV), visna and progressive pneumonia viruses of sheep, Feline Immunodeficiency Virus (FIV), Human Immunodeficiency Viruss (HIV-1 and HIV-2), and Bovine Immunodeficiency Virus (BIV). A preferred lentiviral vector is derived from BIV. Non-limiting examples of BIV vectors are described in WOO1/44458 and the provisional US application titled “Recombinant bovine immunodeficiency virus based gene transfer system” filed on Feb. 4, 2002, which are hereby incorporated by reference, describe an example of a BIV based lentiviral vector system and methods of use. New vector systems are continually being developed to take advantage of particular properties of parent retroviruses or lentiviruses such as host range, usage of alternative cell surface receptors and the like. The present invention is not limited to particular retroviral or lentiviral vectors, but may include any retroviral or lentiviral vector.

In a specific embodiment, a viral vector that contains endostatin-encoding nucleic acid is used. For example, a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. Endostatin-encoding nucleic acid to be used in gene therapy is cloned into the vector, which facilitates delivery of the gene into a patient. Other retroviral vectors, and methods for producing them, are disclosed in international patent publication WO 01/12830.

Adenoviruses are another type of viral vector that can be used in gene therapy.

Adenovirus genomes are linear, double-stranded DNA molecules of approximately 36 kilobase pairs. Each extremity of the viral genome has a short sequence known as the inverted terminal repeat (or ITr), which is necessary for viral replication. The well-characterized molecular genetics of adenovirus render it an advantageous vector for gene transfer. Portions of the viral genome can be substituted with DNA of foreign origin. In addition, recombinant adenoviruses are structurally stable and no rearranged viruses are observed after extensive amplification.

Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety. Additional, generally applicable methods are disclosed in the Examples below.

In a specific embodiment, the nucleic acid to be introduced for purposes of gene therapy using an adenoviral vector comprises an inducible promoter operably linked to the coding region, such that expression of the nucleic acid is controllable by controlling the presence or absence of the appropriate inducer of transcription.

Incorporation of genomic elements into the adenoviral vector may provide for enhanced expression of the DNA sequence encoding a protein such as endostatin. Thus, in accordance with another aspect of the present invention, there is provided an adenoviral vector including at least one DNA sequence encoding an antiangiogenic protein, e.g., endostatin, and at least one genomic element affecting the expression of such DNA sequence. The term “genomic element” is used as previously defined. Such genomic elements include, but are not limited to, introns, the 5′ untranslated region, and the 3′ untranslated region, and portions of the introns and 3′ and 5′ untranslated regions. The adenoviral vector may be as hereinabove described. Promoters which control the DNA sequence may be selected from those described herein and from those known in the art. Preferably the animal is a mammal; more preferably a primate and most preferably a human.

When adenoviral vectors are administered, they may be delivered at a dose of about 1×10⁴ to 1×10⁸ adenoviral vector particles per eye; preferably at a dose of about 1×10⁵ to 1×10⁷ adenoviral vector particles per eye; and most preferably about 1×10⁵ to 1×10⁶ adenoviral vector particles per eye.

The vector, consisting of infectious, but replication-defective, viral particles, which contain at least one DNA sequence encoding an antiangiogenic protein, e.g., endostatin, is administered periocularly in vivo to a host in an amount effective to treat retinal disease in the host. The host may be a mammalian host, including human and non-human primate hosts.

In one embodiment, when administered to a mammalian host, adenoviral vectors are administered in an amount effective to provide an antiangiogenic protein, e.g., endostatin, at levels which are from about 2 to 20 times the basal levels of the antiangiogenic protein found in the tissues of the host.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

Adeno-associated virus (AAV) has also been proposed for use in gene therapy, including endostatin gene therapy for tumors (see, e.g., Nguyen et al., Cancer Research, 58, 5673-5677 (1998)). Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

The viral compositions useful in the practice of the invention may be administered alone or in combination with at least one other agent, such as stabilizing compound, which may be administered in any sterile, biocompatible pharmaceutical carrier, including, but not limited to, saline, buffered saline, dextrose, and water, preferably in physiologically compatible buffers such as Hanks′ solution, Ringer's solution, or physiologically buffered saline. The compositions may be administered to a patient alone, or in combination with other agents, drugs or hormones.

After viral formulations have been prepared, they can be placed in an appropriate container and labeled for treatment of an indicated condition. For administration of viruses encoding endostatin, such labeling would include amount, frequency, and method of administration.

The disclosure of all patents, publications, (including published patent applications), and database accession numbers and depository accession numbers referenced in this specification are specifically incorporated herein by reference in their entirety to the same extent as if each such individual patent, publication, and database accession number, and depository accession number are specifically and individually indicated to be incorporated by reference.

It is understood, however, that the scope of the present invention is not to be limited to the specific embodiments described above. The invention may be practiced other than as particularly described and still be within the scope of the accompanying claims.

### EXAMPLE 1

Generation of Adenoviral Vectors: Method 1

The mouse endostatin (mEndo) cDNA is amplified by polymerase chain reaction (PCR) from mouse collagen XVIII clone ID 748987 from Genome Systems (St. Louis, Mo.) with the primers 5′-ACT GGT GAC GCG GCC CAT ACT CAT CAG GAC TTT CAG CC-3′ (SEQ ID NO:6) and 5′-AAG GGC TAT CGA TCT AGC TGG CAG AGG CCT AT-3′ (SEQ ID NO:7) (598-bp F1 fragment). The mouse immunoglobulin k chain leader sequence (Ig-k leader) is PCR amplified from pSecTag2 (InVitrogen, Carlsbad, Calif.) with the primers 5′-CAC TGC TTA CTG GCT TAT CG-3′ (SEQ ID NO:8)and 5′-CTG ATG AGT ATG GGC CGC GTC ACC AGT GG-3′ (SEQ ID NO:9) (147-bp F2 fragment). PCR is carried out with Pfu DNA polymerase (Stratagene, La Jolla, Calif.) for 35 cycles under the following conditions: 95° C. hot start for 3 min, 95° C. denaturation for 1 min, 55° C. annealing for 1 min, and 72° C. extension for 2 min. The DNA fragments are gel purified. The sig-mEndo chimeric DNA (718 bp) is generated by PCR splice overlap extension with F1 and F2 DNA fragments generated above as templates to assemble mouse Ig-k leader sequence and murine endostatin cDNA. PCR is carried out with the primers 5′-CAC TGC TTA CTG GCT TAT CG-3′ (SEQ ID NO:8) and 5′-AAG GGC TAT CGA TCT AGC TGG CAG AGG CCT AT-3′ (SEQ ID NO: 10), using Pfu DNA polymerase (Stratagene). PCR is run for 35 cycles under the following conditions: 95° C. hot start for 3 min, 95° C. denaturation for 1 min, 60° C annealing for 1 min, and 72° C. extension for 2 min.

The pAvmEndoLxr adenoviral shuttle plasmid is constructed by inserting the 718-bp sig-mEndo chimeric DNA into the NheI and ClaI sites of adenoviral shuttle plasmid, pAvF9lxr, which is downstream of the Rous sarcoma virus (RSV) promoter and upstream of the simian virus 40 (SV40) polyadenylation signal. An AscI and Nhe1 digested simian cytomegalovirus (sCMV) promoter fragment is substituted for the RSV promoter in pAvmEndoLxc, which is otherwise identical to pAvrnEndoLxr. Both shuttle plasmids contain a LoxP site for Cre/lox-mediated recombination. The sequence of the transgenes in the pAvmEndoLxr and pAvrnEndoLxc adenoviral plasmids are confirmed by direct sequencing analysis.

Recombinant Av3mEndo (with E1, E2a, and E3 deleted) encoding the sig-mEndo chimera is generated by Cre/lox-mediated recombination of two plasmids, pSQ3 and pAvmEndoLxr. The pSQ3 plasmid contains a loxP site followed by the Av3 genome with the deletion of the region from the left-end inverted terminal repeat (ITR) to the end of E1a. pAvmEndoLxr and pSQ3 are first linearized with NotI and ClaI restriction enzymes, respectively. A transient transfection is performed with 293 cells (4×10⁵ cells per well of a six-well plate), using the calcium phosphate mammalian transfection system (Promega, Madison, Wis.). The calcium phosphate-DNA precipitate is prepared with 4.8 mg of linearized pAvmEndoLxr, 12 mg of linearized pSQ3, 6 mg of pcmvCre, and 6 mg of pcmvE2a in a total volume of 1.8 ml. A 0.6-ml calcium phosphate-DNA precipitate is added to each well. The 293 cells are incubated with calcium phosphate-DNA precipitate at 37° C. for 16 hr. The precipitate is removed and the cells are washed with phosphate-buffered saline (PBS). Fifteen days posttransfection, cytopathic effect (CPE) is observed. The cells and the medium are then harvested by scraping. The crude viral lysate is prepared by five cycles of freezing and thawing.

The Av3mEndo vector is reamplified in S8 cells with 0.3 mM dexamethasone in Richter's CM containing 5% FBS until CPE is observed. The adenoviral vector titer (particles per milliliter) and biological titer (plaque-forming units [PFU] per milliliter) are determined as described (Mittereder et al., 1996). Recombinant Av3CsmEndo containing sig-mEndo driven by the CMV promoter is generated in the same manner by Cre/lox-mediated recombination of pSQ3 and pAvmEndoLxc. The correct genome structures of the purified Av3mEndo, Av3CsmEndo, and control Av3NulI are confirmed by restriction digests and Southern blot analysis. The Av3mEndo and Av3CsmEndo seedlot are confirmed to be negative for replication-competent adenovirus (RCA).

The supernatant from Av3mEndo-transformed S8 cells contains a 20-kDa protein, the expected size of endostatin, that potently inhibits VEGF165-induced migration of HUVEC cells and ELISA demonstrated that 10⁶ Av3mEndo-transduced Hep3B cells secrete 1-2 μg of murine endostatin per 24 hours.

Generation of Adenoviral Vectors: Method 2

Murine cDNA is obtained by isolating RNA (RNeasy Mini kit; Qiagen, Valencia, Calif.) from snap-frozen 2-week-old C57BL/6 mouse (Charles River Laboratories, Wilmington, Mass.) liver and by treating with Moloney murine leukemia virus reverse transcriptase (Life Technologies, Inc., Gaithersburg, Md.). The murine endostatin gene is cloned into the TA cloning vector (Invitrogen, Carlsbad, Calif.) by PCR using the primers sense 5′-GATCTCTAGACCACCATGCATACTCATCAGGACTT-3′ (SEQ ID NO:11) and antisense 5′-ACTGGAGAAAGAGGTllATCTAGCTACTAG-3′ (SEQ ID NO:12). The 18-amino acid E3/19K signal sequence MRYMILGLLALAAVCSAA (SEQ ID NO:13) is inserted upstream from the endostatin sequence by PCR using the primers sense 5′-GATCTCTAGACCACCATGAGGTACATGATTTTAGGCTTGCTCGCCCTTGCG G CAGTCTGCAGCGCGGCCCATACTCATACTCATCAGGACMTTCAG-3′ (SEQ ID NO:14) and antisense (as above). Plasmid DNA is amplified in DH5 cells (Life Technologies), and the signal sequence-murine endostatin (ss-mEndo) sequence is confirmed (ABI Prism 310 autosequencer; PE Applied Biosystems, Foster City, Calif.).

The ss-mEndo construct is digested with EcoRI and cloned by blunt-end ligation into the multiple cloning site of the adenoviral shuttle plasmid pAd/CMV.1. The resulting plasmid is recombined with type 5 E1A/B-deleted Ad2 and used to infect 293 cells (American Type Culture Collection, Manassas, Va.). Plaque DNA is extracted using proteinase K digestion, phenol extraction, and ethanol precipitation and screened for ss-mEndo by PCR. The resulting virus, Ad-ss-mEndo, is amplified in 293 cells. A similar strategy is used to create control recombinant viruses containing the genes for β-gal (Ad-β-gal) and firefly luciferase (Ad-luc). Viruses are titered using a standard plaque-forming assay in 293 cells. Cells are grown in complete medium consisting of DMEM with 10% FCS, 100 units/ml penicillin, 100 μg/ml streptomycin, 50 μg/ml gentamicin, 0.5 μg/ml Fungizone, and 4 mM glutamine (Biofluids, Rockville, Md.). Cells are infected at MOIs ranging from 0.1 to 100 (105 to 108 pfu per 106 cells in 1.0 ml of complete media) with Ad-ss-mEndo, Ad-luc, or no virus and incubated at 37° C. for 24 h. Supernatants are centrifuged at 2×g for 5 min and assayed for endostatin using a competitive EIA (Cytimmune Sciences, College Park, Md.), according to the manufacturer's instructions. 293 cell supernatants are concentrated 10-fold in cellulose columns (Centricon YM-10; Millipore, Bedford, Mass.) and analyzed by Western blotting (NuPAGE; Novex, San Diego, Calif.) using 570 ng/ml rabbit antimurine endostatin polyclonal IgG antibody (gift of Cytimmune Sciences). The EIA murine endostatin standard is used as a positive control. The susceptibility of the murine colon adenocarcinoma cell line MC38 (developed in the Surgery Branch, National Cancer Institute) to adenoviral infection is tested by infecting cells with Ad-β-gal as described above and assaying for β-gal 24 h later using a staining kit (Boehringer Mannheim, Indianapolis, Ind.). Susceptibility of the murine hepatocyte line NMuLi (American Type Culture Collection) to Ad-β-gal infection is used as a positive control.

Generation of Adenoviral Vectors: Method 3

Liver tissue from a BALB/c mouse is homogenized, and total RNA is extracted (RNeasy kit; Qiagen, Chatsworth, Calif.). First-strand cDNA is amplified by reverse transcription-PCR with oligo(dT) primers (SuperScript II; Life Technologies, Grand Island, N.Y.). The full-length mouse endostatin cDNA is amplified by PCR (sense primer with a ClaI linker, 5′-ATCGATCATACTCATCAGGACTTTCAGCC-3′ (SEQ ID NO: 15); antisense primer with a NotI linker, 5′-GCGGCCGCCTATTTGGAGAAAGAGGTCAT-3′ (SEQ ID NO: 16) for subcloning into pBluescript (Stratagene). A synthetic oligonucleotide coding for the rat insulin leader sequence is cloned in front of the endostatin gene. After sequence confirmation, the rat insulin leader-endostatin cDNA is cloned into the recombinant adenovirus (ADV) shuttle vector pADV.hEF1-α (human elongation factor 1-α) for the rescue of the recombinant adenovirus as described by Bautista, D. S. et al., (1991) Virology 182, 578-596. The viral particles are measured by absorption (A260), and the plaque-forming units are determined by standard agarose-overlay plaque assay on 293 cells. The cDNA for the construction of the ADV.hVEGF165 is obtained through reverse transcription-PCR of RNA isolated from human umbilical vein endothelial cells (HUVEC). JC and LLC cell lines are obtained from American Type Culture Collection. The cells are cultured in RPMI medium 1640 (JC) and DMEM (LLC). All media are supplemented with 10% FBS, 0.2 mM glutamine, and 1% penicillin/streptomycin. HUVEC are isolated from umbilical cords by collagenase type IV (Sigma) perfusion (0.2% in Hanks' balanced salt solution) for 20 min at room temperature. The cells then are cultured on collagen-coated (1% in PBS) plates in M199 medium supplemented with 20% FBS, 0.2 mM glutamine, 1% penicillin/streptomycin, and 1 ng/ml bFGF.

### EXAMPLE 2

### Gene Transfer to Mice and Induction of CNV

Viral vectors are injected into the tail vein of adult C57BL/6 mice. Mice are injected with 2×10¹¹ particles of either Av3mEndo (n=18) or Av3mNull (n=17) or with 6×10¹⁰ particles of either Av3CsmEndo or Av3CsNull. Four days after viral vector injection, the mice are anesthetized with ketamine hydrochloride (100 mg/kg body weight), pupils are dilated with 1% tropicamide, and krypton laser photocoagulation is used to rupture Bruch's membrane at 3 locations in each eye of each mouse as previously described by Tobe, et al. Am. J. Pathol. 153, 1641-1646 (1998). Briefly, krypton laserphotocoagulation (100 μm spot size, 0.1 seconds duration, 120 mW) is delivered using the slit lamp delivery system of a Coherent Model 920 Photocoagulator and a hand held cover slide as a contact lens. Burns are performed in the 9, 12, and 3 o'clock positions 2-3 disc diameters from the optic nerve. Production of a vaporization bubble at the time of laser, which indicates rupture of Bruch's membrane, is an important factor in obtaining CNV, so only burns in which a bubble is produced are included in the study. A bubble is not produced for 1 burn in mice injected with Av3mEndo and 3 burns in mice injected with Av3mNull. The cornea of one eye of a mouse that had been injected with Av3mEndo has a corneal scar that prevented laser use and that eye is not used.

### EXAMPLE 3

### Measurement of the Size of Laser-Induced CNV Lesions

Two weeks after laser treatment, the size of CNV lesions is evaluated by one of two different techniques, measurement of the integrated area of CNV on serial sections as previously reported by Seo, et al., Amer. J. Pathol. 154, 1743-1753 (1999) or measurement of the area of CNV in choroidal flat mounts as described by Edelman et al., Invest. Ophthalmol. Vis. Sci. 41, S834 (2000). For mice injected with Av3mEndo, 10 mice are evaluated by the flat mount technique and 8 by serial sections, and for mice injected with Av3mNull, 10 mice are evaluated by the flat mount technique and 7 by serial sections.

Mice used for the flat mount technique are anesthetized and perfused with 1 ml of phosphate-buffered saline containing 50 mg/ml of fluorescein-labeled dextran (2×10⁶ average mw, Sigma, St. Louis, Mo.) as previously described by Tobe, et al., Invest. Ophthalmol. Vis. Sci. 39, 180-8 (1998). The eyes are removed and fixed for 1 hour in 10% phosphate-buffered formalin. The cornea and lens are removed and the entire retina is carefully dissected from the eyecup. Radial cuts (4-7, average 5) are made from the edge of the eyecup to the equator and the eyecup is flat mounted in Aquamount with the sclera facing down and the choroid facing up. Flat mounts are examined by fluorescence microscopy and images are digitized using a 3 CCD color video camera and a frame grabber. Image-Pro Plus is used to measure the total area of hyperfluorescence associated with each burn, corresponding to the total fibrovascular scar.

For mice injected with Av3mEndo, a total of 19 eyes are evaluated (one eye had a pre-existent corneal scar that precluded laser treatment) and there is one bum that had not been associated with a bubble, so that 56 lesions are measured. For mice injected with Av3mNull, a total of 20 eyes are evaluated and since there are 3 burns that had not been associated with a bubble, 57 lesions are measured. The areas within each eye are averaged and after log transformation, regression analysis with generalized estimating equations (GEE) is performed. This analysis adjusts for correlation between right and left eyes of each mouse.

Mice used to measure the integrated area of CNV on serial sections are sacrificed 2 weeks after laser treatment and eyes are rapidly removed and frozen in optimum cutting temperature embedding compound (OCT; Miles Diagnostics, Elkhart, Ind.). Frozen serial sections (10 μm) are cut through the entire extent of each burn and histochemically stained with biotinylated griffonia simplicifolia lectin B4 (GSA, Vector Laboratories, Burlingame, Calif.) which selectively binds to vascular cells. Slides are incubated in methanol/H₂O₂ for 10 minutes at 4° C., washed with 0.05 M Tris-buffered saline, pH 7.6 (TBS), and incubated for 30 minutes in 10% normal porcine serum. Slides are incubated 2 hours at room temperature with biotinylated GSA and after rinsing with 0.05M TBS, they are incubated with avidin coupled to peroxidase (Vector Laboratories) for 45 minutes at room temperature. After being washed for 10 minutes with 0.05 M TBS, slides are incubated with Histomark Red (Kirkegaard and Perry) to give a red reaction product that is distinguishable from melanin. Some slides are counterstained with Contrast Blue (Kirkegaard and Perry).

To perform quantitative assessments, GSA-stained sections are examined with an Axioskop microscope and images are digitized using a 3 CCD color video camera and a frame grabber. Image-Pro Plus software is used to delineate and measure the area of GSA-stained blood vessels in the subretinal space. For each lesion, area measurements are made for all sections on which some of the lesion appeared and added together to give the integrated area measurement. The measurements within each eye are averaged and regression analysis with GEE is performed.

In initial experiments, the amount of CNV at sites of laser-induced rupture of Bruch's membrane is compared in mice injected with Av3mEndo and mice injected with Av3mNull. The amount of CNV is assessed by two different techniques;

measurement of the area of CNV perfused by fluorescein-labeled dextran on choroidal flat mounts and measurement of the area of CNV on serial sections through the entire lesion. The area of laser-induced CNV in choroidal flat mounts appeared less in mice injected with Av3mEndo compared to uninjected mice or mice injected with Av3Null. The difference seen by visual comparison is confirmed by image analysis performed by investigators masked with respect to treatment group, which showed that the mean area of perfused CNV lesions in mice injected with Av3mEndo is significantly less than that in Av3Null-injected controls (Table 1).

TABLE 1

| | | | | |
|---|---|---|---|---|
| Vector | Mice | Eyes | Lesions | P |

Area of Perfused CNV on Choriodal Flat Mounts

| | | | | | |
|---|---|---|---|---|---|
| | | | | Area (10⁻³ mm²) | |
| Av3mEndo | 10 | 19 | 56 | 13.73 ± 1.36 | <0.0001 |
| Av3Null | 10 | 20 | 57 | 29.41 ± 2.19 | |

Integrated Area of CNV on Serial Sections Through Entire Lesions

| | | | | | |
|---|---|---|---|---|---|
| | | | | Integrated | |
| | | | | Area (10⁻² mm²) | |
| Av3mEndo | 8 | 15 | 44 | 5.88 ± 0.91 | <0.0001 |
| Av3Null | 7 | 13 | 37 | 12.58 ± 2.21 | |

Serial sectioning through CNV lesions also showed smaller lesions in mice injected with Av3mEndo compared to mice injected with Av3Null. The integrated area of CNV obtained by adding together the area of CNV on each serial section, which assesses size in 3 dimensions, confirmed that there is significantly less CNV at sites of Bruch's membrane rupture in mice injected with Av3mEndo compared to Av3Null injected-mice (Table 1). Since both measurement techniques provide very similar information, only choroidal flat mounts are used in subsequent experiments.

There is an inverse correlation between endostatin serum levels and the area of CNV. Serum levels of endostatin are optimal 4-7 days after intravenous injection of the vectors. A group of mice are injected with Av3mEndo, Av3CsmEndo, Av3Null, or Av3CsNull. Laser treatment is done on day 4 and serum is obtained 7 days after injection. With investigators masked with respect to vector group and endostatin serum level, the area of CNV is measured on choroidal flat mounts 14 days after laser photocoagulation. Mice injected with Av3CsmEndo appear to have less CNV than uninjected mice or those injected with Av3CsNull. Image analysis confirms that the area of CNV lesions is significantly less in mice injected with either Av3CsmEndo or Av3mEndo compared to controls (Table 2).

TABLE 2
Area of Perfused CNV on Choriodal Flat Mounts

| Vector | Mice | Eyes | Lesions | Area (10⁻³ mm²) | P |
|---|---|---|---|---|---|
| Av3CsmEndo | 11 | 22 | 66 | 8.87 ± 0.85 | *<0.0001, |
| | | | | | **<0.0001 |
| Av3mEndo | 10 | 19 | 55 | 18.36 ± 2.24 | *0.0013, |
| | | | | | **0.0004 |
| Av3CsNull | 11 | 21 | 62 | 24.41 ± 2.92 | *0.22 |
| Av3Null | 9 | 17 | 48 | 32.91 ± 4.87 | *0.89 |
| No vector | 11 | 21 | 59 | 31.71 ± 3.98 | |

for difference from no vector controls;
**for difference from coresponding null vector control

Plotting the mean area of CNV lesions vs. endostatin serum level in each mouse shows a strong inverse correlation with r=−0.66.

### EXAMPLE 4

### Analysis of Expression of Endostatin in Eye and Liver

To determine whether systemic administration of adenoviral vectors results in significant transduction of the eye, a group of mice is injected with Av3nBg . This vector expresses β-galactosidase from an RSV promoter. After 5 days, the mice are sacrificed and β-galactosidase activity is measured in homogenates of the eye and liver using a chemiluminescence assay. Livers and eyes are snap frozen following removal from mice. On the day of the assay, livers or eyes are homogenized in lysis buffer (40:1 v/v 1× Reporter Lysis Buffer (Promega, Madison Wis.): Protease Inhibitor Cocktail (Sigma, St Louis Mo.)). Protein content is determined by Bradford Assay (Biorad, Hercules Calif.). β-galactosidase activity is determined using the Galacto-Light system (Tropix, Bedford Mass.).

In the livers of mice that received vector, levels of β-galactosidase activity are approximately 1000-fold higher than uninjected controls, whereas in the eye, the levels of this enzyme activity are similar between vector-injected and control animals. The absence of detectable β-galactosidase activity in the eye following administration of an adenovirus expressing this enzyme suggests that the antiangiogenic effect after intravascular injection of endostatin vectors is due to systemically-produced rather than locally-produced endostatin.

### EXAMPLE 5

### Comparison of Mice Injected With Av3mEndo to Those Injected With AV3CsmEndo

Mice are injected in the tail vein with 2×10¹¹ particles of Av3mEndo (n=10) or Av3mNull (n=9), or they are injected with 6×10¹⁰ particles of Av3CsmEndo (n=11) or Av3CsmNull (n=11). A no injection control group (n=11) is also included. Four days after injection, Bruch's membrane is ruptured with laser in three places in each eye of each mouse as described above. Seven days after injection, blood is drawn from the tail vein of each mouse and serum is stored at −80° C. for ELISAs. Eighteen days after injection and 14 days after laser, the area of CNV is assessed on choroidal flat mounts as described above.

Endostatin serum levels are determined with a murine endostatin enzyme-linked immunosobent assay (ELISA) kit (ACCUCYTE murine endostatin:CytImmune Sciences, College Park, Md.) according to the manufacturer's instructions.

Characterization of the second vector construct, Av3CsmEndo, demonstrated that its intravascular injection results in approximately 10-fold higher maximal endostatin levels compared to levels in mice that are injected with the maximum tolerated dose of Av3mEndo particles (2×10¹¹ pfu). Serum levels of endostatin are significantly higher in the Av3mEndo and Av3CsmEndo injected mice than in controls with no injection or a null vector injection. Basal levels of endostatin in mice are found to be between about 30 to 150 ng/ml of serum.

Thus, mice that are injected with a construct in which sig-mEndo expression is driven by the Rous sarcoma virus promoter have moderately high serum levels of endostatin and significantly smaller CNV lesions at sites of laser-induced rupture of Bruch's membrane than mice that are injected with null virus. Mice that are injected with a construct in which sig-mEndo is driven by the simian cytomegalovirus promoter have roughly 10-fold higher endostatin serum levels and have significantly less CNV, with nearly complete inhibition.

### EXAMPLE 6

### Generation of a Recombinant Adenoviral Vector Encoding Human Endostatin

The human endostatin cDNA is PCR amplified from the cDNA of human α1 (XVIII) collagen. The human liver cDNA is generated from human liver poly A RNA (Clonetech, Palo Alto, Calif.) by reverse transcriptase polymerase chain reaction (RT-PCR). The reverse transcription is carried out with the primer of 5′-TTT TTT TTT CAG TGT AAA AGG TC-3′ (SEQ ID NO: 17) using the Perkin Elmer RT-PCR kit (Perkin Elmer Applied Biosystems, Foster City, Calif.) for 1 cycle in the following conditions: room temperature for 10 min, 42° C. reverse transcribing for 3 min, 99° C. denaturation for 5 min, 5° C. cooling for 5 min, and hold at 4° C. until the cDNA is ethanol precipitated and resuspended. The 790 bp human endostatin cDNA fragment is PCR amplified from the prepared cDNA with the primers of 5′-CAG ATG ACA TCC TGG CCA G-3′ (SEQ ID NO: 18) and 5′-CTA TAC AGG AAA GTA TGG CAG C-3′ (SEQ ID NO: 19). PCR is carried out for 35 cycles in the following condition: 95° C. hot start for 3 min, 80° C. for 3 min followed by the addition of Pfu DNA polymerase (Stratagene, La Jolla, Calif.), 95° C. denaturation for 1 min, 55° C. annealing for 1 min. and 72° C. extension for 3 min. The 790 bp human endostatin cDNA fragment is gel purified and reamplified as described except using the annealing temperature of 58° C. The 790 bp human endostatin cDNA fragment is gel purified and cloned into PCR-Script Amp SK+ using PCR-Script Cloning Kits (Stratagene) according to the manufacturer's procedure to generate pcrhend 1. The human endostatin cDNA region of the pcrhend I plasmid is confirmed with the direct sequencing analysis by Gene Therapy Core Technologies Molecular Core Laboratory at Genetic Therapy, Inc. Gaithersburg, Md.

The human endostatin cDNA fragment is assembled with human BM40 basement protein leader according to the following procedure. The BM40 basement protein leader is generated by annealing 2 pieces of synthesized oligonucleotides, 5′-GCC AAG CTT CCA TGA GGG CCT GGA TCT TCT TTC TCC TTT GCC TGG CCG GGA GGG CTC TGG CAG CCC CTC AGC AAG AAG CGC TCG CTC ACA GCC ACC GCG ACT TCC AGC CGG TGC TCC A-3′ (sense) (SEQ ID NO:20), and 5′-CCA GGT GGA GCA CCG GCT GGA AGT CGC GGT GGC TGT GAG CGA GCG CTT CTT GCT GAG GGG CTG CCA GAG CCC TCC CGG CCA GGC AAA GGA GAA AGA AGA TCC AGG CCC TCA TGG AAG CTT GGC-3′ (antisense) (SEQ ID NO:21) followed by Hind III and Sex A1 digestion. The digested BM40 basement protein leader is cloned into Hind III and Sex A1 sites of pcrhend 1 to generate pBmpcrhen plasmid. The entire sig-hEndo region of the pBmpcrhen plasmid is confirmed with the direct sequencing analysis.

The adenovial shuttle plasmid pAV1bmhend1x is generated by substitution of the Factor IX (F9) containing sequence with the sig-Endo containing sequence in pAvF9Lxr adenoviral shuttle plasmid in the following procedure. An 800 bp fragment containing sig-hEndo sequence is generated from pBmpcrhen by SacI digestion followed by Klenow fill in and Sal I digestion. The pAvF9Lxr plasmid is digested with Bam HI restriction enzyme followed by Klenow fill in and digested with Sal I restriction enzyme to remove F9 containing sequences. The two digested fragments are gel purified and ligated to generate pAV1bmhendlx.

Human endostatin cDNA is RT-PCR generated from the C-terminus of cDNA of human al (XVIII) collagen from human liver poly A RNA. The human BM40 basement protein leader is generated from two pieces of synthesized oligonucleotides. The annealed human BM40 basement protein leader is cloned 5′ of the human endostatin cDNA to generate sig-hEndo chimeric protein for the secretion of human endostatin protein. The sig-hEndo chimeric DNA is cloned into the adenoviral shuttle plasmid, pAvF9lxr to create pAV1bmhendlx (FIG. 12A). The entire sig-hEndo chimeric sequence is confirmed by auto sequencing analysis.

Recombinant Av3brnhendlx (with E1, E2a, and E3-deletions) encoding the sig-hEndo chimeric protein is generated by the “Quick Cre/Lox two plasmid system” according to the following procedure. The plasmids pAV1bmhendlx and pSQ3 are linearized first with Not I and Cla I restriction enzymes, respectively. The S8 cells are pretreated with 0.3 μM dexamethasone 24 hours before the transient transfection that is performed on the 6-well plate at 4×105 S8 cells per well using LipofectAMINE PLUS Reagent (Life Technologies, Rockville, Md.). The lipofectamine complexed DNA is prepared with 1 μg of linearized pSQ3, 0.5 μg pCre, and 0.5 μg linearized pAV1bmhendlx, and 6 μl of lipofectamine according to the manufacturer's procedure (Life Technologies). The S8 cells are incubated with lipofectamine complexed DNA at 37° C. for 4.5 hours. The lipofectamine complexed DNA is removed and the cells are ished with PBS. The transfected S8 cells are cultured at 37° C. with 5% CO2 until the cytopathic effect is observed. The cells and the medium are harvested by scraping. The crude viral lysate is prepared by five cycles of freezing and thawing. The Av3bmhendlx is re-amplified in S8 cells with 0.3 μM dexamethasone in Richter's CM medium containing 5% FBS until cytopathic effect is observed.

Av3bmhendlx-mediated human endostatin expression and secretion is characterized in vector-transduced S8 cells. The supernatant protein of cells infected with Av3bmhendlx, i.e., human endostatin, is analyzed by SDS-PAGE. Each 20 μg of supernatant protein is analyzed on 4 to 12% linear gradient precasted gel. The SDS-PAGE is transferred to a polyvinylidene fluoride membrane. The membrane is stained with Coomassie blue R-250. 20 kDa protein bands, corresponding to the correct size of human endostatin, are excised from a membrane blot and subjected to N-terminal protein sequencing analysis. The protein sequence of three major secreted proteins is determined, with 50% containing the amino acid sequence of human endostatin with the additional amino acid residues APQQEALA (SEQ ID NO: 5), 25% containing residues LA, and 25% containing no residues from human BM40 basement protein signal peptide. The 20 kDa protein is not found in the supernatant protein from Av3NulI cells. The results demonstrate that S8 cells transduced with Av3bmhendlx express and secrete human endostatin after it is processed from human BM40 basement protein signal peptide.

### EXAMPLE 7

### Inhibition of Retinal Leakage, Thickening, Detachment, and Neovascularization In Vivo by BIV Vector Mediated Anti-Angiogenesis Gene Expression

Bovine immunodeficiency viral (BIV) vectors encoding eGFP are generated from the three component system (described in published international patent application number WO0144458, the disclosure of which is incorporated by reference herein in its entirety). The transfer vector BIVendostatin is derived from pBSV4MGpptGAG, a BIV-based transfer vector construct encoding eGFP under MND U3 promoter according to Takahashi, K. et al. Hum. Gene Ther. 13, 1305-1316 (2002). The eGFP coding sequence is replaced with murine endostatin (mEndo). Specifically, to delete eGFP from the parent plasmid pBSV4MGpptGAG, the eGFP plus some flanking sequence is amplified by PCR. The primers used are: eGFP1FOR 5′-GCGCATGTCGACAGAATATGGGCCAAAC-3′, which incorporated a SalI site to the 5′ end of the PCR product, and eGFP1REV 5′-GCGCTACTGCAGAGCTAATGAGCTACAC-3′, which incorporated a PstI site to the 3′ end of the PCR product. This fragment is cut with SalI and PstI and ligated with pBSH(KS+) which is also previously digested with SalI and PstI, creating pBS2eGFP. The ExSite PCR-Based Site-Directed Mutagenesis Kit (Stratagene, LaJolla, Calif.) is used to delete eGFP from pBS2eGFP. PCR primers are designed that flanked the outer portions of the eGFP gene, and pointed outward, thus amplifying everything, including the entire flanking sequence and plasmid, except eGFP. The primers used are: DELeGFP1FOR: 5′-CCGGCTAGCTTAAGGGTGGCGACCGGT-3′, which added NheI and AfllI restriction sites, and DELeGFP1REV: 5′-GCTTCGAACGCGTAGCGGCCAACCCTC-3′, which added BstBI and MluI restriction sites. The amplicon is treated according to the manufacturer's instruction, and ligated to form pBS2deleGFP with eGFP deleted but with the flanking sequence from the parental plasmid remaining. This strategy also created four new restriction sites in the middle. This fragment is sequenced to ensure no mutations had been introduced. The mEndo gene insert is prepared from an adenoviral shuttle plasmid, pAVmEndolxr according to Mori, K. et al. Amer. J. Pathol. 159, 313-320 (2001). This plasmid is digested with NheI and ClaI to release the mEndo fragment. This fragment is ligated with pBS2deleGFP, which is previously digested with NheI and BstBI (compatible ends with ClaI), generating pBS2deleGFPmEndo. The Woodchuck Hepatitis Virus post-transcriptional regulatory element (WPRE) is inserted downstream of mEndo at the MluI site, creating pBS2deleGFPmEndoPRE. Finally, the plasmid pBS2deleGFPmEndoPRE is digested with Bsu36I and BbvCI to release the mEndo coding sequence and the orignal eGFP flanking sequences, and this fragment is ligated with pBS4MGpptGAG which is previously digested with Bsu36I and BbvCI, generating pBvMNDmEndoPRE. A null BIV vector, pBvMNDPRE, is also generated and served as a negative control vector. PBvMNDPRE is identical to pBvMNDmEndPRE except for the absence of the mEndo coding sequence.

Briefly, to generate BIV vector particles encoding mEndo (BIVendostatin), 293T cells in 150 mm dish (2×10⁷ cells/dish) are transfected with 45 μg pBvMNDmEndoPRE, 45 μg BIV-based packaging construct pBIVminipack, and 13.5 μg pseudotyping envelope expression construct pVSV-G. To generate BIV null vectors (BIVNull), pBvMNDEndoPRE is replaced with pBvMNDPRE construct. Forty eight hours post-transfection, the vector is harvested and filtered through a 0.45 μm filter. The vector supernatant is then concentrated by ultracentrifugation. The concentrated vector is aliquoted and stored at −80° C. until used.

The concentrated vector is assayed for reverse transcriptase (RT) activity, a measure of vector particles. Both BIVendostatin and BIVNull vectors scored RT activity of approximately 15 μg per ml. We have previously shown with a BIV-based vector encoding eGFP, that one ng of RT equals approximately 1×10⁵ transducing units (T.U.). Therefore, the estimated titer of BIVendostatin vector is 1.5×10⁹ T.U./ml. To examine if the BIVendostatin vector could mediate efficient production of mEndo, Cf2Th cells in a 6-well plate (4×10⁵ cells/well) are transduced with either 1 μl (15 ng RT equivalent vector particles) of BIVNull vector or the same amount of BIVendostatin vector. Forty eight hours post-transduction, the cell supernatant is assayed for endostatin expression using a commercial endostatin assay kit, Accucyte Mouse Endostatin assay system, following the manufacturer's instruction (Cytimmune Sciences Inc., College Park, Md.). BIVendostatin vector-transduced cells produced 412 ng/ml of endostatin while the BIVNull vector did not score detectable level of endostatin.

A BIV vector encoding murine endostatin prepared according to O'Reilly et al., Cell;88(2):277-85 (1997) is administered via subretinal injection of singly transgenic mice (IRBP/rtTA-TRE/VEGF tgMICE) that express Vascular Endothelial Growth Factor from mouse photoreceptor cells upon induction with Doxycyclin. BIV vectors are injected into mouse right eyes while the left eyes serve as controls without injection of vectors. Three weeks after vector injection. 0.5 mg/ml of Doxycyclin is placed in the drinking water for the transgenic mice. It is found that Doxycyclin-induced VEGF expression results in severe neovascularization on the left eyes of the transgenic mice by examination of fluorescein angiograms. VEGF-induced neovascularization is completely blocked by BIV vector-mediated endostatin expression in the right eyes in the same animals.

Immunohistochemically stained ocular frozen sections from the eyes of adult C57BL/6 mice given subretinal injections of 1.5×106 transducing units (TU) of BIVendostatin in the right eye and 1.5×106 TU of BIVNull in the left eye, euthanized four weeks after vector injection showed that eyes injected with BIVendostatin show heavy staining for endostatin in RPE cells and throughout the inner nuclear layer with dense staining of the walls of some blood vessels. Linear stained structures in the inner plexiform layer are typical of Muller cell processes. Eyes injected with BIVNull showed reaction product along the internal limiting membrane and Bruch's membrane which is likely to be due to cross-reactivity with collagen XVIII

Adult double transgenic rho/rtTA-TRE/VEGF mice given a subretinal injection of 1.5×106 transducing units (TU) of BIVendostatin in the right eye and 1.5×106 TU of BIVNull in the left eye and started on 2 mg/ml of doxycycline in their drinking water four weeks after vector injection are assayed for retinal vascular permeability is measured using [3H]mannitol after 3 days on doxycycline. The retina to lung (RLLR) is significantly reduced in eyes expressing endostatin compared to fellow eyes that are injected with null vector, indicating that expression of endostatin with either of 2 different vector systems results in suppression of VEGF-induced vascular permeability in the retina.

Adult rho/rtTA-TRE/VEGF mice, given subretinal injections of 1.5×106 TU of BIVendostatin in the right eye and 1.5×106 TU of BIVNull in the left eye and started on 0.5 mg/ml of doxycycline in their drinking water four weeks after vector injection are subjected to fluorescein angiography four days after initiation of doxycycline. Mice injected with BIVendostatin show normal appearing retinal vessels with distinct walls indicating little or no permeation of fluorescein in BIVendostatin-injected eyes, while BIVNull-injected eyes show diffuse fluorescence throughout the retina indicating extensive extravasation of fluorescein. Seven days after initiation of doxycycline, there is still little evidence of fluorescein leakage in BIVendostatin-injected eyes, compared to massive leakage in BIVNull-injected eyes.

When retinal vascular permeability is increased, fluid collects in the retina resulting in retinal thickening. Thickening in the macula is called macular edema and there is an inverse correlation between amount of abnormal thickening and visual acuity. Thus, retinal thickening is a physiologically relevant variable to assess and lends itself to precise quantitation using in vivo imaging techniques such as optical coherence tomography (OCT) or retinal thickness analysis (RTA). Seven days after starting 0.5 mg/ml of doxycycline in the drinking water to induce VEGF expression, frozen sections of BIVendostatin-injected eyes show less retinal thickening than BIVNull-injected eyes. Eight rho/rtTA-TRE/VEGF mice are used to quantitatively assess the effect of endostatin on VEGF-induced retinal thickness. Four weeks after subretinal injection of 1.5×106 TU of BIVendostatin in the right eye and 1.5×106 TU of BIVNull in the left eye, mice are started on 0.5 mg/ml of doxycycline in their drinking water. Seven days after starting doxycycline, retinal thickness is measured by image analysis 300 μm from each edge of the optic nerve along the horizontal meridian and averaged to give a single experimental value in each eye. Mean retinal thickness is significantly greater in BIVNull-injected eyesthan in BIVendostatin-injected eyes

Four weeks after subretinal injection of 1.5×106 TU of BIVendostatin in the right eye and 1.5×106 TU of BIVNull in the left eye, mice are started on 0.5 mg/ml of doxycycline in their drinking water. Seven days after starting doxycycline, the amount of retinal neovascularization is significantly greater in BIVNulI-injected eyes compared to BIVendostatin-injected eyes.

When rho/rtTA-TRE/VEGF mice are given 2 mg/ml of doxycycline in their drinking water for 5 days or longer, they express high levels of VEGF and develop severe neovascularization and retinal detachment. Eleven rho/rtTA-TRE/VEGF mice are used to assess the effect of BIV-vectored endostatin on this severe phenotype resulting from high levels of retinal VEGF. Four weeks after subretinal injection of 1.5×106 TU of BIVendostatin in the right eye and 1.5×106 TU of BIVNull in the left eye, mice are started on 2 mg/ml of doxycycline in their drinking water. Seven days after starting doxycycline, mice are euthanized and eyes are fixed in 2% paraformaldehyde. After gross pathologic examination, they are frozen in OCT and sectioned. Serial sections are stained with hematoxylin and eosin. Observers masked with respect to vector group determine whether each eye had a total, partial, or no retinal detachment. There are significantly fewer total retinal detachments in eyes injected with BIVendostatin than eyes injected with BIVNull. Four BIVendostatin-injected eyes have no retinal detachment compared to only one BIVNull-injected eye.

### EXAMPLE 8

### Intraocular Injections

For studies using the adenoviral vectors, adult mice are given a subretinal injection of 6×10⁷ particles of a 1:1 mixture of AGVC7mEndo and AGVas521 in one eye and 6×10⁷ particles of AGVnull in the other eye. For studies using the lentiviral vectors, the mice received 5×10⁶ transducing units (TU) of BIVendostatin in one eye and. 5×10⁶ TU of BIVNull in the other eye. Pulled glass micropipets are calibrated to deliver 1 μl of vehicle upon depression of a foot switch. Injections are performed using a condensing lens system on the dissecting microscope and a contact lens, which allowed visualization of the retina during the injection. The mice are anesthetized, pupils are dilated, and under a dissecting microscope, the sharpened tip of the micropipet is passed through the sclera posterior to the limbus and is positioned just above the retina. Depression of the foot switch caused the jet of injection fluid to penetrate the retina. This technique is very atraumatic and the direct visualization allows confirmation that the injection is successful, because of the appearance of a small retinal detachment (bleb). The blebs are quite uniform in size and involved slightly less than half of the retina.

### EXAMPLE 9

### Gutless Adenoviral Vector-Mediated Regulated Endostatin Expression in the Eye

Regulated expression of endostatin in vivo using an adenoviral vector delivery system is achieved according to methods described by Xu et al., Molecular Therapy; 3:262 (2001). The AGV vectors encoding the tamoxifen-inducible chimeric transcription factor, AGVas521, and the regulatable endostatin transgene, AGVC7mEndo, are generated from the plasmids, pAGVas521 and pAGVC7mEndo, respectively. In addition to the transgene expression cassette (see below), the AGV plasmids contain the left and right ITRs flanked by unique Pac I sites, the packaging signal of Ad5, and approximately 25 kb of the human alpha synuclein intronic region as a DNA “stuffer” according to Reddy, P. S. et al., Molec. Ther. 5, 63-73 (2002). The plasmid pAGVas521, contains the tamoxifen-inducible chimeric transcription factor composed of the unique zinc finger DNA binding domain, a modified ligand binding domain based on the human estrogen receptor, and the transactivating region derived from VP16 driven by the CMV promoter. To construct pAGVas521, the chimeric transcription factor expression cassette is isolated from pAvCv-C7LBD by digestion with Nru I and Bam HI, and inserted into pBLSV2. The plasmid pBLSV2 is derived from pBluescript (Strategene, La Jolla, Calif.), and contains two multicloning site polylinkers. The resulting plasmid, pBLSV2as521 is digested with Bspe I and ligated to pGTI.24aPL2 digested with Xma I, to generated pGTI24as521. pGTI24aPL2 contains a multicloning site polylinker flanked by human synuclein stuffer DNA. Next, pGTI24as521 is digested with Pac I to liberate the plasmid backbone, and combined with Pme I-Mlu I digested pBV2, usinghomologous recombination in BJ5138 E. coli according to Toietta, G. et al., Mol. Ther. 5, 204-210 (2002), to generate pAGVas521. The plasmid pBV2 contains 26625 bp of human synuclein stuffer DNA.

The plasmid pAGVC7mEndo contains the ligand-inducible transcription factor regulated promoter driving the expression of murine endostatin. To construct pAGVC7mEndo, the plasmid pav-6X2C7tatamendo is digested with Asc I (blunted) and Bam HI and inserted into pBLSV2C7endo. The plasmid pBLSV2C7endo is digested with Bam HI and Eco RI, ends filled in, and ligated to pGTI245.aPL2 digested with Sma I to generated pGTI24C7endo. Finally, pGTI245.aPL2 is digested with Pac I to liberate the plasmid backbone, and combined with Pme I and Mlu I digested pBV4, using homologous recombination in BJ5138 E. coli per Toietta, supra, to generate pAGVC7mEndo. The plasmid pBV4 contains 27191 bp of human synuclein stuffer DNA.

Gutless vector generation and large scale production and purification are performed as described by Reddy et al. The particle titers are determined by optical density measurements. DNA extracted from CsCI-purified vectors are analyzed by restriction enzyme digestions to verify vector integrity. A hexon-based quantitative PCR assay is used to determine the level of helper virus contamination in AGV preparations. Helper contamination levels of AGVNull, AGVas521, and AGVC7mEndo preparations used in this study are 0.09%, 1.9%, and 1.4%, respectively.

This tamoxifen-inducible system displays high-level inducible endostatin expression in the serum of C57BL/6 mice following systemic administration of early generation, E1/E2a/E3-deficient vectors encoding either the inducible transcription factor, or the regulatable endostatin transgene.

This regulatory system is composed of two components, an inducible transcription factor, and a responsive promoter driving expression of mouse endostatin. The transcription factor consists of a modified human estrogen ligand binding domain that is responsive to tamoxifen, a unique cysteine 2-histidine 2 zinc finger DNA binding motif, and a minimal transactivation domain from VP 16. The responsive promoter consists of 6 repeats of the DNA sequence recognized by the transcription factor DNA binding domain (DBD) and a DNA encoding endostatin. In the presence of tamoxifen, this transcription factor activates transcription from a unique target nucleic acid sequence linked to a minimal promoter. When evaluated with a luciferase reporter in vitro, tamoxifen induces expression up to 250 fold. This system is incorporated into two gutless adenoviral vectors, which are devoid of all viral coding regions. One vector encodes the transcription factor, and the second encodes the target promoter driving transcription of a nucleic acid encoding endostatin. The two vectors are injected into mice, which results in efficient liver transduction. Administration of tamoxifen to the mice results in inducible expression of endostatin, yielding extremely high plasma levels of up to 20 ug/ml. Tamoxifen induction is achieved four times over a two-month period. In the absence of tamoxifen, background levels of endostatin are observed.

Mice injected with the vector pair of AGVC7mEndo and AGVas521 constituting the inducible system treated with tamoxifen showed prominent staining for endostatin throughout the entire retina, demonstrating strong induction of endostatin expression in the retina.

Retinal vascular permeability is measured using [3H]mannitol as tracer in adult IRBP/rtTA-TRE/VEGF mice given a subretinal injection of 6×107 particles of a 1:1 mixture of AGVC7mEndo and AGVas521 in the right eye and 6×107 particles of AGVNull in the left eye, followed by treatment with tamoxifen for six days to induce endostatin expression, the last 3 of which the mice also received doxycycline to induce VEGF expression. Both the retina to lung (RLLR) and retina to renal leakage ratios (RRLR) are significantly reduced in eyes with induced expression of both endostatin and VEGF compared to fellow eyes that had induced expression of VEGF alone. This demonstrates that endostatin suppresses VEGF-induced increased permeability of retinal vessels.

### EXAMPLE 10

### Transgenic Mice and Assay Methods

Two lines of double transgenic mice with inducible expression of VEGF in the retina have been generated by Ohno-Matsui, K. et al. Am. J. Pathol. 160, 711-719 (2002) are used in this study. In one of the lines, the interphotoreceptor retinoid binding protein (IRBP) promoter is combined with the reverse tetracycline transactivator (rtTA) system to direct doxycyline-inducible expression of VEGF in photoreceptors. These are referred to as IRBP/rtTA-TRE/VEGF mice. In the second line of double transgenic mice, the rhodopsin promoter rather than the IRBP promoter is combined with the reverse tetracycline transactivator system to direct doxycyline-inducible expression of VEGF in photoreceptors. These are referred to as rho/rtTA-TRE/VEGF mice.

Immunohistochemistry for Endostatin

Eyes are punctured and placed in 4% paraformaldehyde/5% sucrose and then incubated overnight in 0.1 M phosphate buffer, pH 7.4 for 1.5 hours at 4° C. Eyes are then rinsed and rapidly frozen in a 2:1 mixture of 0.1 M phosphate buffer/20 % sucrose in OCT. Ten μm frozen sections are dried and post-fixed in cold 4 % paraformaldehyde for 30 minutes. After rinsing, slides are blocked with cold methanol containing 6.25% H2O2 for 15 minutes and then with 2% skim milk in Tris-buffered saline (TBS) for 30 minutes at room temperature. Slides are incubated in 1.5 μg/ml of polyclonal goat IgG directed against mouse endostatin (R&D Systems, Minneapolis, Minn.) in 2% milk/TBS for 1 hour at room temperature. After washing in 0.1% milk/TBS for 10 minutes, slides are incubated 30 minutes at room temperature in 2 μg/ml biotin-conjugated anti-goat IgG (Santa Cruz Biotechnology, Santa Cruz, Calif.) in 2% milk/TBS. After washing 10 minutes in 0.1% milk/TBS, slides are incubated for 30 minutes at room temperature in streptavidin-phosphatase (Kirkegaard and Perry, Cabin John, Md.). After 3 five minute washes in 0.05 M Tris HCl, slides are developed with HistoMark Red (Kirkegaard and Perry) and mounted.

Measurement of retinal vascular permeability using [³H]mannitol as tracer

Adult double transgenic IRBP/rtTA-TRE/VEGF mice (n=11) are given a subretinal injection of 6×10⁷ particles of a 1:1 mixture of AGVC7mEndo and AGVas521 in the right eye and 6×10⁷ particles of AGVNull in the left eye. The next day mice are started on daily intraperitoneal injections of 500 μg of tamoxifen in 5% DMSO in sunflower oil and after 3 days, they are given 2 mg/ml of doxycycline in their drinking water. Three days after initiating doxycycline, retinal vascular permeability is measured using [³H]mannitol. Briefly, mice are given an intraperitoneal injection of 1 μCi/gram body weight of [3H]mannitol (New England Nuclear, Boston, Mass.). After one hour, mice are sacrificed and eyes are removed. The cornea and lens are removed and the entire retina is carefully dissected from the eyecup and placed within pre-weighed scintillation vials. The thoracic cavity is opened and the left superior lobe of the lung is removed and placed in another pre-weighed scintillation vial. A left dorsal incision is made and the retroperitoneal space is entered without entering the peritoneal cavity. The renal vessels are clamped with a forceps and the left kidney is removed, cleaned of all fat, and placed into a pre-weighed scintillation vial. All liquid is removed from the vials and remaining droplets are allowed to evaporate over 20 minutes. The vials are weighed and the tissue weights are recorded. One ml of NCSII solubilizing solution (Amersham, Chicago, Ill.) is added to each vial and the vials are incubated overnight in a 50° C. water bath. The solubilized tissue is brought to room temperature and decolorized with 20% benzoyl peroxide in toluene in a 50° C. water bath. The vials are brought to room temperature and 5 ml of Cytoscint ES (ICN, Aurora, Ohio) and 30 μl of glacial acetic acid are added. The vials are stored for several hours in darkness at 4° C. to eliminate chemoluminescence. Radioactivity is counted with a Wallac 1409 Liquid Scintillation Counter (Gaithersburg, Md.).

Assessment of retinal vascular permeability using fluorescein leakage

Adult rho/rtTA-TRE/VEGF double transgenic mice are given subretinal injections of 1.5×106 transducing units (TU) of BIVendostatin in the right eye and 1.5×106 TU of BIVNull in the left eye. Four weeks after vector injection, mice are started on 0.5 mg/ml of doxycycline in their drinking water. Four or 7 days later, mice are given an intraperitoneal injection of 12 μl/g body weight of 1% fluorescein sodium (Alcon, Fort Worth, Tex.) and after 1 minute pictures are taken of each eye using in vivo fluorescence microscopy. Seven days after initiating VEGF expression, another mouse is euthanized and retinal frozen sections are cut through the posterior part of the retina adjacent to the optic nerve in the same location in each eye. The sections are stained with Griffonia simplicifolia lectin, hematoxylin, and eosin. The retina in the BIVNull injected eye (F and H) is much thicker than the retina in the BiVendostatin-injected eye (E and G).

Measurement of Retinal Thickness

Adult rho/rtTA-TRE/VEGF double transgenic mice are given subretinal injections of 1.5×106 TU of BIVendostatin in the right eye and 1.5×106 TU of BIVNull in the left eye. Four weeks after vector injection, mice are started on 0.5 mg/ml of doxycycline in their drinking water. Seven days after initiating doxycycline, mice are euthanized and 10 μm retinal frozen sections are cut through the posterior part of the retina adjacent to the optic nerve in the same location in each eye. The sections are stained with biotinylated Griffonia simplicifolia lectin B4 (GSA, Vector Laboratories, Burlingame, Calif.), which selectively binds to vascular cells. Slides are incubated in methanol/H2O2 for 10 minutes at 4° C., washed with 0.05 M Tris-buffered saline, pH 7.6 (TBS), and incubated for 30 minutes in 10% normal porcine serum. Slides are incubated 2 hours at room temperature with biotinylated GSA and after rinsing with 0.05M TBS, they are incubated with avidin coupled to peroxidase (Vector Laboratories) for 45 minutes at room temperature. The slides are developed with HistoMark Red (Kirkegaard and Perry) to give a red reaction product and counter stained with hematoxylin and eosin. Retinal thickness is measured by image analysis 300 μm from each edge of the optic nerve and averaged to give a single experimental value.

Assessment of retinal neovascularization in double transgenic mice

Three rho/rtTA-TRE/VEGF mice are used to assess the effect of endostatin on VEGF-induced retinal neovascularization. Four weeks after subretinal injection of 1.5×106 TU of BIVendostatin in the right eye and 1.5×106 TU of BIVNull in the left eye, mice are started on 0.5 mg/ml of doxycycline in their drinking water. Seven days after starting doxycycline, mice are euthanized and each eye is sectioned (10 μm sections) from the peripheral edge of the iris to the other peripheral edge 180° away. Sections are stained with Griffonia simplicifolia lectin (GSA), hematoxylin, and eosin.

The area of GSA staining in the photoreceptor layer is an indication of the amount of neovascularization and is measured on sections 100 μm apart from one edge of the iris to the other edge (generally 13 sections per eye). The average of these 13 measurements constitutes a single experimental value, the area of neovascularization per section.

Assessment of Retinal Detachment in Double Transgenic Mice

Eleven adult rho/rtTA-TRE/VEGF double transgenic mice are given subretinal injections of 1.5×106 TU of BIVendostatin in the right eye and 1.5×106 TU of BIVNull in the left eye. Four weeks after vector injection, mice are started on 2 mg/ml of doxycycline in their drinking water. After 4 days, mice are anesthetized, pupils are dilated and funduscopic examination is performed on each eye, by two independent observers noting if there is a total or partial retinal detachment, or no detachment.

### EXAMPLE 11

### Periocular Injections of Vectors and Expression in Retina and RPE

Adult C57BL/6 mice are injected periocularly with adenoviral vectors encoding pigment epithelium-derived factor (PEDF) or soluble VEGF receptor (sFlt-1). PEDF and sFlt-1 are detected in retinas of mice injected with the vectors encoding the factors. When a vector encoding beta-galactosidase (AGVcnBg) is injected, at early time points there is no intraocular staining, but eight weeks or longer after periocular injections, focal areas of LacZ staining are seen in the retina and retinal pigment epithelium.

What is claimed is:

1. A method for delivering a protein to the retina of a subject in need of such delivery, comprising periocularly injecting the individual with an effective amount of a viral vector comprising a protein-encoding nucleic acid.

2. The method of claim 1 wherein the protein is an endostatin.

3. The method of claim 2, wherein the endostatin is a polypeptide fragment of the polypeptide with the amino acid sequence set forth in SEQ ID NO:1, a derivative of the polypeptide with the amino acid sequence set forth in SEQ ID NO:1, or a variant of the polypeptide with the amino acid sequence set forth in SEQ ID NO:1.

4. The method of claim 3, wherein the viral vector is selected from the group consisting of an adenovirus, an adeno-associated virus, a retrovirus, and a lentivirus.

5. The method of claim 4, wherein the viral vector is an adenoviral vector.

6. The method of claim 1, wherein the protein is a member selected from the group consisting of soluble vascular endothelial growth factor receptor, pigment epithelium-derived factor, angiostatin (plasminogen fragment), rod-derived cone viability factor, antiangiogenic antithrombin III, cartilage-derived inhibitor (CDI), CD59 complement fragment, fibronectin fragment, Gro-beta, a heparinase, human chorionic gonadotropin (hCG), an interferon, interferon inducible protein (IP-10), interleukin-12, kringle 5 (plasminogen fragment), metalloproteinase inhibitors (TIMPs), placental ribonuclease inhibitor, plasminogen activator inhibitor, platelet factor-4 (PF4), prolactin 16 kD fragment, proliferin-related protein (PRP), thrombospondin-1 (TSP-1), transforming growth factor-beta (TGF-b), vasculostatin, and vasostatin (calreticulin fragment).

7. The method of claim 6, wherein the viral vector is selected from the group consisting of an adenovirus, an adeno-associated virus, a retrovirus, and a lentivirus.

8. The method of claim 7, wherein the viral vector is an adenoviral vector.

9. The method of claim 4, wherein the viral vector is a lentiviral vector.

10. The method of claim 7, wherein the viral vector is a lentiviral vector.

11. The method of claim 9, wherein the lentiviral vector is derived from a bovine immunodeficiency virus.

12. The method of claim 10, wherein the lentiviral vector is derived from a bovine immunodeficiency virus.
